(19) United States
(12) Patent Application Publication
(10) Pub. No.: US 2021/0139836 A1
(43) Pub. Date: May 13, 2021

(54) AGGREGATED CELL MASS DISPERSING DEVICE AND METHOD OF DISPERSING AGGREGATED CELL MASS

(71) Applicant: JSR Corporation, Tokyo (JP)

(72) Inventors: Daichi SUEMASA, Tokyo (JP); Eiji HAYASHI, Tokyo (JP)

(73) Assignee: JSR Corporation, Tokyo (JP)

(21) Appl. No.: 17/091,145

(22) Filed: Nov. 6, 2020

(30) Foreign Application Priority Data
Nov. 8, 2019 (JP) 2019-203235

(51) Int. Cl. C12M 1/26; C12M 1/00; B01F 11/00; B01F 15/00

(52) U.S. Cl. CPC C12M 33/04; C12M 23/26; B01F 2215/0073; B01F 11/0082; B01F 15/00772; B01F 11/0074

(57) ABSTRACT

An aggregated cell mass dispersing device (100) of the present invention includes: a storage container (101) storing a liquid (L) containing an aggregated cell mass; a pipette (102) whose tip is pushed against an inner bottom surface (101a) of the storage container; and an elastic body (103) supporting an outer bottom surface of the storage container.

### BACKGROUND OF THE INVENTION

### Field of the Invention

The present invention relates to an aggregated cell mass dispersing device and a method of dispersing an aggregated cell mass.

Priority is claimed on Japanese Patent Application No. 2019-203235, filed on Nov. 8, 2019, the content of which is incorporated herein by reference.

### Description of Related Art

It is known that a dispersion operation of aggregated cell masses, which is performed in the process of cell culture, is remarkably affected by a difference in the technique of each operator. The term “dispersion operation of aggregated cell masses” means that cells forming an aggregated cell mass are detached from each other by allowing the aggregated cell mass to pass through a narrow space to make the aggregated cell mass dispersed to be one to ten single cells or small aggregated cell masses. The space though which the aggregated cell mass passes is formed between the tip of a pipette and a bottom surface of a microtube in a state in which the pipette is inserted into the microtube, and the tip thereof is brought into close contact with the bottom surface of the microtube. The insertion angle of the pipette and the accuracy of force with which the pipette is brought into close contact with the bottom surface largely depend on the technique of each operator, and there is a problem that the size of the formed space and furthermore the dispersion efficiency of the aggregated cell masses vary from operator to operator. It is required to automate the operation of dispersing such aggregated cell masses in order to stabilize quality of cultured cells. Techniques relating to the automation of the dispersion of the aggregated cell masses are disclosed in United States Patent Application, Publication No. 2018/0080002, United States Patent Application, Publication No. 2017/0159003, and United States Patent Application, Publication No. 2017/0191019.

### SUMMARY OF THE INVENTION

The distance between the tip of the pipette and the bottom surface of a storage container may vary due to wear of the tip of the pipette or the like, and in this case, it is necessary to finely adjust the position to which the pipette is pushed according to the variation. However, it is difficult to perform this fine adjustment during the process of the automated operation of dispersing aggregated cell masses, and it is necessary to perform the operation of dispersing aggregated cell masses manually. As a result, the dispersion of the aggregated cell masses may be less efficient.

The present invention has been made in view of the above circumstances, and an object is to provide an aggregated cell mass dispersing device capable of dispersing an aggregated cell mass with high efficiency stably, a method of dispersing an aggregated cell mass, and a method of collecting an aggregated cell mass.

In order to achieve the above object, the present invention employs the following means.

(1) An aggregated cell mass dispersing device according to an aspect of the present invention includes: a storage container storing a liquid containing an aggregated cell mass; a pipette whose tip is pushed against an inner bottom surface of the storage container; and an elastic body supporting an outer bottom surface of the storage container.

(2) In the aggregated cell mass dispersing device according to (1), the elastic body may be a spring disposed to extend and contract in a direction parallel to a direction in which the pipette is pushed, and one end of the spring may be fixed to the outer bottom surface of the storage container.

(3) In the aggregated cell mass dispersing device according to (1), the elastic body may be provided with a first support member supporting the storage container at one side and a spring disposed to extend and contract in a direction parallel to a direction in which the pipette is pushed, and one end of the spring may be fixed to the other side of the first support member.

(4) In the aggregated cell mass dispersing device according to any one of (1) to (3), the elastic body may be further provided with a second support member to which the other end of the spring is fixed and which supports the spring.

(5) In the aggregated cell mass dispersing device according to any one of (1) to (4), the pipette preferably becomes thinner toward the tip.

(6) In the aggregated cell mass dispersing device according to any one of (1) to (5), a section on a bottom of the storage container, to which the tip of the pipette is pushed, is preferably recessed toward the elastic body side.

(7) In the aggregated cell mass dispersing device according to any one of (1) to (6), the storage container may be one or more tubes whose bottom side is blocked, and which extends opposite to the bottom to be installed in a stand manner.

(8) In the aggregated cell mass dispersing device according to (7), the aggregated cell mass dispersing device preferably further includes a guide member surrounding an outer peripheral portion of the tube.

(9) A method of dispersing an aggregated cell mass using the aggregated cell mass dispersing device according to any one of (1) to (8) according to an aspect of the present invention includes: a liquid storage step of storing a liquid containing an aggregated cell mass in the storage container; a pipette pushing step of pushing the tip of the pipette against the inner bottom surface of the storage container; and a pipette pulling step of pulling the tip of the pipette away from the inner bottom surface of the storage container.

(10) In the method of dispersing an aggregated cell mass according to (9), the pipette pushing step and the pipette pulling step are preferably repeated 50 times or more alternately.

In the aggregated cell mass dispersing device of the present invention, the storage container is supported by the elastic body, and even though pushing force of the pipette is too large, the elastic body absorbs part of the force. Therefore, it is possible to prevent an excessive pressure from being applied to the bottom of the storage container, and to prevent the bottom of the storage container from being damaged by the pressure. Since there is no concern that the bottom of the storage container is damaged and there is no need to reduce the pushing force of the pipette, the pipette can be pushed until the pipette is brought into close contact with the bottom. Therefore, it is possible to avoid the problem such as insufficient pushing force. Therefore, even in a case in which the distance between the tip of the pipette and the bottom surface of the storage container varies due to wear of the pipette tip or the like, it is not necessary to adjust the position to which the pipette is pushed according to the variation. Thus, it is possible to perform a pipetting operation automatically. Since magnitude of the force for pushing the pipette, a direction, and the like are uniformized by automation, the aggregated cell mass can be dispersed into a desired size with high efficiency stably.

### BRIEF DESCRIPTION OF THE DRAWINGS

FIG. 1A is a perspective view of an aggregated cell mass dispersing device according to a first embodiment of the present invention.

FIG. 1B is a partially-enlarged view of the aggregated cell mass dispersing device in FIG. 1A.

FIG. 2A is a view illustrating a method of dispersing an aggregated cell mass according to the first embodiment of the present invention.

FIG. 2B is a view illustrating the method of dispersing an aggregated cell mass according to the first embodiment of the present invention.

FIG. 2C is a view illustrating the method of dispersing an aggregated cell mass according to the first embodiment of the present invention.

FIG. 3 is a perspective view of an aggregated cell mass dispersing device according to a second embodiment of the present invention.

FIG. 4 is a perspective view of an aggregated cell mass dispersing device according to a third embodiment of the present invention.

FIG. 5A is an image showing a dispersed state of aggregated cell masses.

FIG. 5B is an image showing a dispersed state of the aggregated cell masses.

FIG. 5C is an image showing a dispersed state of the aggregated cell masses.

FIG. 5D is an image showing a dispersed state of the aggregated cell masses.

FIG. 5E is an image showing a dispersed state of the aggregated cell masses.

FIG. 5F is an image showing a dispersed state of the aggregated cell masses.

FIG. 5G is an image showing a dispersed state of the aggregated cell masses.

FIG. 5H is an image showing a dispersed state of the aggregated cell masses.

FIG. 6 is a graph showing a size variation of the aggregated cell masses during a dispersion process.

FIG. 7 is a graph showing a variation in the number of cells for the aggregated cell masses in the dispersion process.

### DETAILED DESCRIPTION OF THE INVENTION

Hereinafter, an aggregated cell mass dispersing device and a method of dispersing an aggregated cell mass according to an embodiment to which the present invention is applied will be described in detail with reference to the drawings. There are cases in which the drawings used in the following description are shown with enlargement of their characteristic parts for purposes of expediency in order to facilitate understanding of features, and the dimensional proportions and the like of the various components are not necessarily the same as the actual one. In addition, in the following description, the materials and dimensions are exemplary examples, and the present invention is not limited thereto; however, appropriate modifications can be made within a range not departing from the scope of the present invention.

### First Embodiment

FIG. 1A is a perspective view schematically showing a configuration of an aggregated cell mass dispersing device 100 according to a first embodiment of the present invention. FIG. 1B is an enlarged view showing a region R surrounded by alternate long and short dash line in the aggregated cell mass dispersing device 100 of FIG. 1A. The aggregated cell mass dispersing device 100 includes a storage container 101, a pipette 102, and an elastic body 103.

The storage container 101 is a vessel for storing a liquid L containing aggregated cell masses. In order to enhance an effect of pushing the pipette 102 against the aggregated cell mass, a microtube having a small bottom area is preferably used as the storage container 101. Here, a case in which a plurality of microtubes each of which has a blocked bottom side and extends opposite to the bottom (extends to an upper side) to be installed in a stand manner is provided as an exemplary example.

A section on the bottom of the storage container 101, to which a tip 102a of the pipette is pushed, is preferably recessed toward the elastic body 103 side. In this case, the tip 102a of the pipette can be inserted into a recessed section (recess portion) and supported by a side wall of the recess portion, so that the stability of an angle at which the pipette 102 is pushed can be enhanced.

The pipette 102 is installed such that the tip 102a faces an inner bottom surface 101a of the storage container and a distance between the tip 102a and the inner bottom surface 101a can be freely adjusted, and at least the pipette 102 can be pushed against the inner bottom surface.

The pipette 102 preferably becomes thinner toward the tip 102a. In this case, a pressure to push the pipette 102 against the aggregated cell mass can be increased, and a dispersion efficiency of the aggregated cell mass can be increased. The inner diameter of an opening portion of the tip 102a is preferably 0.5 mm or higher and 10 mm or lower. In a case in which the inner diameter is lower than 0.5 mm, it is difficult to pull up dispersed cells. In a case in which the inner diameter higher than 10 mm, an aggregated cell mass that is not dispersed is pulled into the pipette 102 and the dispersion efficiency decreases.

The elastic body 103 supports an outer bottom surface 101b of the storage container. The elastic body 103 is provided with a first support member 104 that supports the storage container 101 at one side (upper side) 104a and a spring 105 that is disposed to extend and contract in the operation direction (pushing direction and pulling direction) D of the pipette 102. One end 105a of the spring is fixed to the other side (lower side) 104b of the first support member 104.

The elastic body 103 is preferably further provided with a second support member 106 to which the other end 105b of the spring is fixed and which supports the spring 105. In this case, stability when pushing the pipette 102 can be improved.

The spring constant of the spring 105 is preferably 10 N/m or higher and 50,000 N/m or lower. In a case in which the spring constant is lower than 10 N/m, a sufficient repulsive force generated by the elastic body 103 cannot be obtained, and the force for pushing the pipette 102 is weakened. As a result, the dispersion of the aggregated cell masses is disturbed. On the other hand, in a case in which the spring constant is higher than 50,000 N/m, a repulsive force of the elastic body 103 is too large, so that the effect of suppressing the excessive pressure applied to the bottom of the storage container decreases.

FIGS. 1A and 1B illustrate a case in which the springs 105 are installed at four positions so that corners of the first support member 104 and corners of the second support member 106 are connected to face each other. The spring 105 serves as supporting the storage container 101 through the first support member 104. Therefore, the springs 105 are symmetrically disposed so that the first support member 104 and the storage container 101 are not tilted, that is, the repulsive force of the springs 105 uniformly acts on the entirety of the first support member 104. Specifically, for example, in a plan view from a moving direction D of the pipette, it is preferable that a plurality of springs are arranged at equal intervals on circles drawn at positions equidistant from the center of the first support member 104.

FIGS. 2A to 2C are views illustrating a method of dispersing an aggregated cell mass using the aggregated cell mass dispersing device 100 described above. The method of dispersing an aggregated cell mass mainly includes the following steps.

Firstly, as shown in FIG. 2A, a liquid L containing aggregated cell masses is stored in the storage container 101 (liquid storage step). In this case, the tip 102a of the pipette is spaced from the inner bottom surface 101a of the storage container, and the pipette is in a state of standing by inside the storage container 101.

Next, as shown in FIG. 2B, the tip 102a of the pipette is pushed against and brought into close contact with the inner bottom surface 101a of the storage container (pipette pushing step). However, a space S (not shown) having a size about a dispersed single cell is provided between the tip 102a of the pipette and the inner bottom surface 101a of the storage container. Alternatively, the tip 102a of the pipette is pushed against and brought into close contact with the inner bottom surface 101a of the storage container to form a space S between the tip 102a of the pipette and the inner bottom surface 101a of the storage container, the space S having a size about a dispersed single cell. Thereby, cells attached to the aggregated cell mass are detached from the aggregated cell mass passing through the space S by contacting with the opening portion of the pipette 102, so that the remained single cell mass can be pulled into the pipette.

Next, as shown in FIG. 2C, the tip 102a of the pipette is pulled away from the inner bottom surface 101a of the storage container, and the aggregated cell mass that is not dispersed is allowed to flow into a position to which the pipette 102 is pushed (pipette pulling step). The pipette 102 is pushed again when the aggregated cell masses have gathered at the position to which the pipette 102 is pushed, and the pipette 102 is pulled away when the aggregated cell masses in the space S have been dispersed.

Then, until each of the cell sizes in a storage container 102 becomes a single cell size, the pipette pushing step and the pipette pulling step are alternately repeated. Thereby, the aggregated cell masses can be dispersed. The number of repetitions depends on the amount of the liquid L stored in the storage container 101, but the pipette pushing step and the pipette pulling step are preferably repeated 50 times or more in order to disperse all of the aggregated cell masses in the storage container 101 into single cells.

As described above, in the aggregated cell mass dispersing device 100 of the present embodiment, the storage container 101 is supported by the elastic body, and even though the force for pushing the pipette is too large, the elastic body absorbs part of the force. Therefore, it is possible to prevent an excessive pressure from being applied to the bottom of the storage container 101, and to prevent the bottom of the storage container 101 from being damaged by the pressure. Since there is no concern that the bottom of the storage container 101 is damaged and there is no need to reduce the pushing force of the pipette, the pipette can be pushed until the pipette is brought into close contact with the bottom. Therefore, it is possible to avoid the problem such as insufficient pushing force. Therefore, even in a case in which the distance between the tip of the pipette and the bottom surface of the storage container varies due to wear of the pipette tip or the like, it is not necessary to adjust the position to which the pipette is pushed according to the variation. Thus, it is possible to perform a pipetting operation automatically. Since the magnitude of the force for pushing the pipette, the direction, and the like are uniformized by automation, the aggregated cell masses can be dispersed into the single cells with high efficiency stably.

FIG. 3 is a perspective view schematically showing a configuration of an aggregated cell mass dispersing device 200 according to a second embodiment of the present invention. In the aggregated cell mass dispersing device 200, a plurality of tube-shaped (cylindrical) vessels are used as the storage container 102, and a guide member 107 that surrounds an outer peripheral portion thereof is further provided. Other configurations are the same as the configurations of the aggregated cell mass dispersing device 100 of the first embodiment, and portions corresponding to the aggregated cell mass dispersing device 100 are denoted by the same reference numerals regardless of the difference in shape.

The guide member 107 includes a plate-shaped member 108 having a through-hole 108a in a thickness direction and a third support member 109 that supports the plate-shaped member 108. A plurality of tube-shaped storage containers 102 are installed such that the storage containers 102 are communicated with the through-holes 108a one by one, and bottoms of the storage containers 102 are brought into close contact with an elastic body 103. An inner diameter of the through-hole 108a is about the same as an outer diameter of the storage container 102, so that the storage container 102 communicated with the through-hole 108a is fixed so as not to tilt.

According to the configuration of the second embodiment, the tube-shaped storage container 102 that easily falls can be vertically installed in a stand manner with respect to the elastic body 103 that is the support member. Therefore, since an insertion angle of the pipette against the storage container 102 is almost fixed, it is possible to eliminate one of causes of the size variation of the space through which the aggregated cell mass passes.

FIG. 4 is a perspective view schematically showing a configuration of an aggregated cell mass dispersing device 300 according to a third embodiment of the present invention. In the aggregated cell mass dispersing device 300, the elastic body 103 is provided without the first support member 104, and the bottom of the storage container is directly supported by the spring 105. Other configurations are the same as the configurations of the aggregated cell mass dispersing device 100 of the first embodiment, and portions corresponding to the aggregated cell mass dispersing device 100 are denoted by the same reference numerals regardless of the difference in shape.

The spring 105 is disposed to extend and contract in a direction (moving direction D) parallel to the direction in which the pipette is pushed, and one end 105a is fixed to the outer bottom surface 101b of the storage container. FIG. 4 further illustrates a case in which the other end 105b of the spring is connected to and supported by the plate-shaped second support member.

In the aggregated cell mass dispersing device 100 of the first embodiment and the aggregated cell mass dispersing device 200 of the second embodiment, the repulsive force from the spring 105 uniformly acts on any of the storage containers 102 by providing the first support member 104. Therefore, for example, even in a case in which the tip of the pipette 102 to be inserted into a part of the storage container 101 is not brought into close contact with the bottom of the storage container 101 due to a dimensional error, the repulsive force acting on the storage container 102 is difficult to strengthen locally. On the other hand, in the aggregated cell mass dispersing device 300 of the third embodiment, any of the storage containers 102 are configured to directly receive the repulsive force by the spring 105, and thus, it is possible to apply the repulsive force required for bringing the tip of the pipette into close contact with the bottom of the storage container locally.

### Example

Hereinafter, the effects of the present invention will be made more apparent by examples. The present invention is not limited to the following example, and appropriate modifications can be made within a range not departing from the scope of the present invention.

Aggregated cell masses were dispersed by using the above described aggregated cell mass dispersing device and method of dispersing an aggregated cell mass. Forty-eight microtubes were used as the storage container, and 10 μl of a liquid containing the aggregated cell masses was stored in each of the microtubes. The pipette was pushed against and pulled away from the inner bottom surface of the storage container automatically and pushing and pulling the pipette was repeatedly performed 195 times for 10 minutes.

FIGS. 5A to 5H are images illustrating dispersion states of the aggregated cell masses in a case in which the number of repetition times of pushing and pulling the pipette (the number of pipetting times) is set to 0, 15, 45, 75, 105, 135, 165, and 195, respectively.

FIG. 6 is a graph showing the size variation of aggregated cell masses during a dispersion process, based on FIGS. 5A to 5H. The horizontal axis of the graph shows the number of repetition times of pushing and pulling the pipette. The vertical axis of the graph shows an average size of the aggregated cell masses in all of the storage containers. It can be seen that as the pipette is repeatedly pushed and pulled, the aggregated cell masses are more dispersed, and the size is reduced. Note that, there is almost no size variation in a case in which the number of repetitions is 50 times or higher, which indicates that the aggregated cell masses have been dispersed into cells of a minimum unit.

FIG. 7 is a graph showing the variation of the number of cells during a dispersion process, based on FIGS. 5A to 5H. The horizontal axis of the graph shows the number of repetition times of pushing and pulling the pipette. The vertical axis of the graph shows the number of cells contained in all of the storage containers. It can be seen that as the pipette is repeatedly pushed and pulled, the aggregated cell masses are more dispersed, and the number of cells is monotonically increased.

From the results shown in FIGS. 5 to 7, it can be seen that by automatically performing the pipetting operation, the aggregated cell masses can be dispersed stably with high efficiency and at high speed. The reason for the stable dispersion even though the pipette tip position is not adjusted due to the automation of the pipetting operation is that the effect of the pipette tip displacement is suppressed by the repulsive force of the elastic body.

### EXPLANATION OF REFERENCES

100, 200, 300 aggregated cell mass dispersing device
101 storage container
101a inner bottom surface of storage container
101b outer bottom surface of storage container
102 pipette
102a tip of pipette
103 elastic body
104 first support member
104a one side of first support member
104b the other side of first support member
105 spring
105a one end of spring
105b the other end of spring
106 second support member
107 guide member
108 plate-shaped member
108a through-hole
109 third support member
D moving direction of pipette
L liquid
R region
S space

1. An aggregated cell mass dispersing device, comprising:
a storage container storing a liquid comprising an aggregated cell mass;
a pipette whose tip is pushed against an inner bottom surface of the storage container; and
an elastic body supporting an outer bottom surface of the storage container.

2. The aggregated cell mass dispersing device of claim 1,
wherein the elastic body is a spring disposed to extend and contract in a direction parallel to a direction in which the pipette is pushed, and
one end of the spring is fixed to the outer bottom surface of the storage container.

3. The aggregated cell mass dispersing device of claim 1,
wherein the elastic body is provided with a first support member supporting the storage container at one side and a spring disposed to extend and contract in a direction parallel to a direction in which the pipette is pushed, and
one end of the spring is fixed to the other side of the first support member.

4. The aggregated cell mass dispersing device of claim 1, wherein the elastic body is provided with a second support member to which an end of a spring is fixed and which supports the spring.

5. The aggregated cell mass dispersing device of claim 1, wherein the pipette becomes thinner toward the tip.

6. The aggregated cell mass dispersing device of claim 1, wherein a section on a bottom of the storage container, against which the tip of the pipette is pushed, is recessed toward the elastic body side.

7. The aggregated cell mass dispersing device of claim 1, wherein the storage container is one or more tubes whose bottom side is closed, and which is provided upright so as to extend opposite to the bottom.

8. The aggregated cell mass dispersing device of claim 7, further comprising a guide member surrounding an outer peripheral portion of the one or more tubes.

9. A method of dispersing an aggregated cell mass using the aggregated cell mass dispersing device of claim 1, the method comprising:
storing a liquid comprising an aggregated cell mass in the storage container;
pushing the tip of the pipette against the inner bottom surface of the storage container; and
pulling the tip of the pipette away from the inner bottom surface of the storage container.

10. The method of claim 9, wherein the pushing and the pulling are repeated 50 times or more alternately.
